(19) United States
(12) Patent Application Publication
(10) Pub. No.: US 2016/0038101 A1
(43) Pub. Date: Feb. 11, 2016

(54) PATIENT COUCH SUPPORT, PATIENT COUCH AND SYSTEM FOR DISSIPATING HEAT OF THE PATIENT COUCH SUPPORT

(71) Applicants: Thomas Benner, Erlangen (DE); Katrin Wohlfarth, Fellbach (DE)

(72) Inventors: Thomas Benner, Erlangen (DE); Katrin Wohlfarth, Fellbach (DE)

(21) Appl. No.: 14/818,724

(22) Filed: Aug. 5, 2015

(30) Foreign Application Priority Data
Aug. 6, 2014 (DE) 102014215544.4

(51) Int. Cl. A61B 6/04; A47C 21/04

(52) U.S. Cl. CPC A61B 6/045; A47C 21/044

(57) ABSTRACT

One or more embodiments provide a patient couch support for dissipating heat of the patient couch support. The patient couch support includes a surface to accept an examination object and at least one channel for carrying a fluid running at least partly inside the patient couch support. The at least one channel has at least one inlet opening for delivering the fluid and at least one outlet opening for taking away the fluid. One or more embodiments provide a corresponding patient couch and a system for dissipating heat of the patient couch support.

### CROSS-REFERENCE TO RELATED APPLICATIONS

The present patent document claims the benefit of DE 102014215544.4, filed on Aug. 6, 2014, which is hereby incorporated by reference in its entirety.

### TECHNICAL FIELD

The present embodiments relate to a patient couch support for dissipating heat of the patient couch support. In addition, the present embodiments relate to a corresponding patient couch and a system for dissipating heat of the patient couch support.

### BACKGROUND

In medical imaging, examination objects such as human or animal patients are frequently supported on a patient couch support that is placed on or in a patient couch, and examination objects are brought into the imaging device lying on the patient couch. In computed tomography (CT) and magnetic resonance tomography (MRT), the patient couch also serves to move the examination object into an examination bore. In an examination or operation (e.g., such as a cardiological intervention using x-ray fluoroscopy) that lasts for an extended time, the temperature on the support surface of the examination object increases (e.g., in the patient's back area when the patient is lying on their back), because of the lack of ventilation. This may be unpleasant for the examination object and may promote transpiration. During an MRT examination, one way the transpiration effect is promoted is by increased RF energy emission into the body of the examination object during imaging with high specific absorption rate (SAR), known as a high SAR scan. Additional heat may result from a spinal column coil (e.g., through so-called eddy current hot-spots). The consequence of temperature increases may include increased patient movements that can lead to movement artifacts in the images obtained, or increased moisture caused by sweating that may represent a safety problem or may cause image errors (e.g., through so-called fold-in artifacts).

Ventilators are currently used to avoid or reduce a heating up of the contact surface between the examination object and the patient couch support or patient couch. For example, ventilators blow cooling air into an examination bore. The air supply may be controlled or regulated manually by an operator, or automatically by a regulation device. A disadvantage of ventilators is that only the surface of the examination object lies in the cooling air flow.

### SUMMARY AND DESCRIPTION

One or more embodiments provide an improved a patient couch support that dissipates the heat from the patient couch support that an examination object is lying on better than previously known patient couch supports. Further, one or more embodiments provide a corresponding patient couch and a system for dissipating heat of the patient couch support.

An embodiment provides a patient couch support for dissipating heat of the patient couch support including a surface for receiving an examination object, at least one channel for guiding a fluid running at least partly in the interior of the patient support, at least one inlet opening for supplying the fluid and at least one outlet opening for taking away the fluid.

The patient couch support may be disposed on or in a patient couch and is designed to take up and dissipate heat from the patient couch support on the surface that an examination object is lying, and cooling the contact surface between examination object and patient couch support. The patient couch support has at least one channel that runs at least partly inside the patient couch support. The channel is suitable for carrying a fluid (e.g., a liquid or gas). The fluid carried through the channel has a lower temperature than the surface of the examination object and is in thermal contact with the surface. A temperature equalization process takes place and the contact surface cools down. In addition to heat introduced by the examination object into the patient couch support, thermal energy of the patient couch support (e.g., caused by the patient couch, by a previous heating up or by the heating up of the patient couch support through an imaging process or other influences) may also heat up the patient couch support. The fluid carried through the patient couch support may reduce this thermal energy of the patient couch support. The channel has at least one inlet opening for supplying the fluid and at least one outlet opening for taking away the fluid. For example, the inlet opening and the outlet opening may be disposed between the patient couch support and a patient couch (e.g., in the middle between the patient couch support and the patient couch). Accommodating the examination object may also be understood as including a part of the patient (e.g., the head of a human patient) being accommodated by the patient couch support. The surface of the patient couch support may have a rectangular footprint. For example, a rectangular footprint may have dimensions such that a human patient lying on their back may be supported on the patient couch support. The course of the channel is adapted to the position of the examination object to be supported (e.g., such as a patient lying on their back or lying on their side), and to the examination object.

In an embodiment, the at least one inlet opening and the at least one outlet opening are each disposed in an edge area of the surface of the patient couch support.

By disposing the inlet openings and output openings in an edge area of the surface of the patient couch support, the openings are easily accessible.

In an embodiment, the at least one channel is formed by cutouts in a foam material.

In this embodiment, the patient couch support may be a foam material with gulleys or channels let into it. The raised structures help to support the examination object while the cutouts carry the heat-dissipating fluid. In a patient couch support that is a foam material and that has a channel structure, a more rigid foam material may be more useful than in a patient couch support without channels, in order to avoid an undesired deep depression of the material and constriction of the channel structure under the weight of an examination object. The necessary stiffness may be determined by mechanics or computer simulations.

In an embodiment, the density of the channel run of the channel may be predetermined over the surface of the patient couch support.

A channel run may be understood as a section of a channel or of a number of channels. For example, if a number of channel are in parallel, then the density of the channel runs of the channels over the surface of the patient couch support is calculated by the quotients of the number of channel runs and the cross-sectional length (e.g., 20 runs per meter). A density profile over the surface of the patient couch support may be provided. For example, a patient couch support that is designed for a patient lying on their back, a higher density of channel runs may be provided over the surface of the patient couch support in the area of the spinal column than provided in the edge area. For patient couch supports that are developed for other patent positions (e.g., such as a patient lying on their side), or other examination areas (e.g., such as the patient's head area), other channel run densities may be provided so that the density of the channel runs of the at least one channel over the surface of the patient couch support depends on a predeterminable support position of the examination object and/or a predeterminable examination area of the examination object.

In an embodiment, the at least one channel has a serpentine form.

A serpentine form refers to an arrangement or a course of the at least one channel in loops. A serpentine form may enable a large surface of the patient couch support to be covered with a few channels (e.g., in the extreme case with a single channel), that may increase the efficiency of the heat dissipation or the cooling.

In an embodiment, the at least one channel has a flat spiral form.

A spiral form (e.g., an Archimedean spiral form or a Fermat spiral form) also may enable a large surface of the patient couch support to be covered with a few channels or with a single channel. The spirals do not have to have the same aspect ratio. Depending on the examination object to be cooled, the spirals may be elongated (e.g., in order to be adapted into a rectangular footprint of the patient couch support).

The patient couch support may include a number of channels (e.g., that at least partly run in a straight line).

Channels running in a straight line may have the advantage that the channels may easily have air flow through the channels (e.g., generated by a fan) that has no direct feed to inlet and outlet openings. For example, this approach may be used for a magnetic resonance tomography device with a fan that blows air into the examination bore.

In an embodiment, the channels have a herringbone or ear of corn pattern.

For example, a herringbone pattern or ear of corn pattern is a structure that is reminiscent of the bones (e.g., with or without backbone) of a fish, or ripening stages of an ear of corn. Herringbone and ear of corn structures are provided because air may flow easily through the structures.

In an embodiment, at least one inlet opening may accept the fluid from a pumping device and/or at least one outlet opening may output the fluid to the pump.

If the fluid involves a liquid, then the pumping device may involve a known pump. If the fluid involves a gaseous fluid (e.g., such as air), the pumping device may be a ventilator or a fan. For a liquid fluid, both the outlet openings and the inlet openings may be connected to the pumping device in order to pump the liquid in the channel or into the channel, and to take the liquid out of the channel or out of the channels.

In an embodiment, at least one channel may be open on a side for accepting the examination object.

In this embodiment, the at least one channel has the form of an open gulley, a groove, a joint gap or a trough, suitable if the fluid is gaseous (e.g., air). The at least one channel is disposed so that it is covered by an examination object supported on the patient couch support. The open channels are closed off by the examination object, through which the thermally-conducting fluid is in thermally-conducting contact with the examination object, but the fluid may not escape through the open side. To improve hygiene, a cloth is arranged between the examination object and an open channel. The open channel may be suitable for a patient couch support with a channel that is ventilated and exhausted by a fan or by a naturally occurring air circulation. This embodiment may be referred to as a passive approach.

In an embodiment, the at least one channel is disposed within the patient couch support.

A channel within the patient couch support may guarantee that the at least one channel (e.g., having the form of a tube or a hose) is only open at the inlet openings and the outlet openings, or is designed to introduce and/or remove the fluid. Because heat transfer through this system may occur through the material of the patient couch support (e.g., even when it is a thin design), a flow of fluid may be forced through the at least one channel (e.g., by a pump device). This approach may be referred to as an active approach.

The at least one channel may have only one input opening for supplying the fluid and precisely one output opening for taking away the fluid.

The patient couch support may include only one channel. The channel has a serpentine form or spiral form because a large surface of the patient couch support has channel runs passing through it.

The input opening and the output opening are disposed in the edge area of the same side of the surface of the patient couch support.

In an arrangement with the input opening and the output opening on the same side of the surface of the patient couch support, less space for any connections to the inlet opening and/or to the outlet opening may be needed. Furthermore, the inlet opening and the outlet opening may be arranged on one side that, on introduction into the examination bore, is the furthest from the examination bore opening. Using this arrangement, the danger of a collision with edge areas of the examination bore may be reduced.

In an embodiment, the patient couch support may include a local magnetic resonance coil device.

In magnetic resonance imaging, local magnetic resonance coil devices may be needed and may be disposed in the immediate vicinity of the examination area to be examined. The combination of a local magnetic resonance coil device and patient couch support with heat dissipation may be advantageous in relation to usage of space and proximity to heat sources.

In an embodiment, the local magnetic resonance coil device is a spinal column coil or a head coil.

A spinal column coil and a head coil are examples for a magnetic resonance coil devices.

In an embodiment, a patient couch for dissipating heat of an examination object is provided. The patient couch includes one of the previously described patient couch supports.

Frequently, patient couches are designed to be used with patient couch supports. For example, patient couches may have a shape that the patient couch support can be laid into to make a form fit. Furthermore, a patient couch may have cutouts that the inlet opening and the outlet opening of the patient couch support can be accepted into. A patient couch support may also be integrated into a patient couch so that the patient couch support and the patient couch form one unit.

In an embodiment, a system for dissipating heat of an examination object includes one of the previously described patient couches and a pumping device for at least delivering the fluid into the inlet opening, and the fluid has a predeterminable throughput and/or a predeterminable temperature.

If the fluid involves a gas (e.g., such as air), then the pumping device may deliver the fluid into the inlet opening for the fluid to flow through the channel or the channels, and to flow out through one or more outlet openings into the environment. As an alternative (e.g., in the case of a liquid fluid), the pumping device may receive the fluid again at an outlet opening. The pumping device may be disposed in the patient couch (e.g., in a foot of the patient couch). The cooling effect may be adjusted by providing a pre-specified a throughput (e.g., in liters per hour) and/or a pre-specified temperature (e.g., in degrees Celsius). Temperature and/or throughput may be adjusted manually (e.g., by a user) or automatically (e.g., by an adjustment device, such as a computer). When an adjustment device is used, regulation methods are known using a measurement of an actual temperature, a specification of a desired temperature and a regulation strategy for reaching the desired temperature may be used.

### BRIEF DESCRIPTION OF THE DRAWINGS

FIG. 1 depicts a patient couch support according to the prior art.

FIG. 2 depicts an example schematic of a patient couch support with a herringbone pattern depicted from above.

FIG. 3 depicts an example schematic of the patient couch support from FIG. 2 depicted in a cross-section.

FIG. 4 depicts an example schematic of a patient couch support with a channel running in a serpentine shape depicted from above.

FIG. 5 depicts an example schematic of the patient couch support from FIG. 4 depicted in cross-section.

FIG. 6 depicts an example schematic of a patient couch support with a channel running in a spiral shape depicted from above.

FIG. 7 depicts an example schematic of the patient couch support from FIG. 6 depicted in a cross-section.

FIG. 8 depicts an example schematic of a system for dissipating heat of an examination object with a patient couch support with a channel running in a spiral shape depicted from above.

FIG. 9 depicts an example schematic of the patient couch support from FIG. 8 depicted in a cross section.

### DETAILED DESCRIPTION

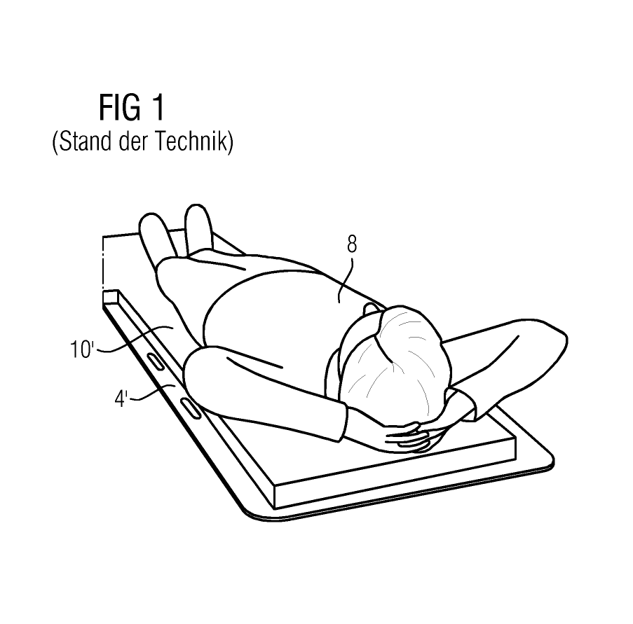

FIG. 1 depicts a patient couch support 10′, according to the prior art. The patient couch support 10′ (e.g., with a rectangular footprint) is disposed on a patient couch 4′. Supported on the patient couch support 10′ is an examination object 8 (e.g., a human patient lying on their back). Through the contact of the back of the examination object 8 with the patient couch support 10′ and the dissipation of heat by the examination object 8, the contact surface is heated up. Sweat may form (e.g., that may have a negative effect on the imaging).

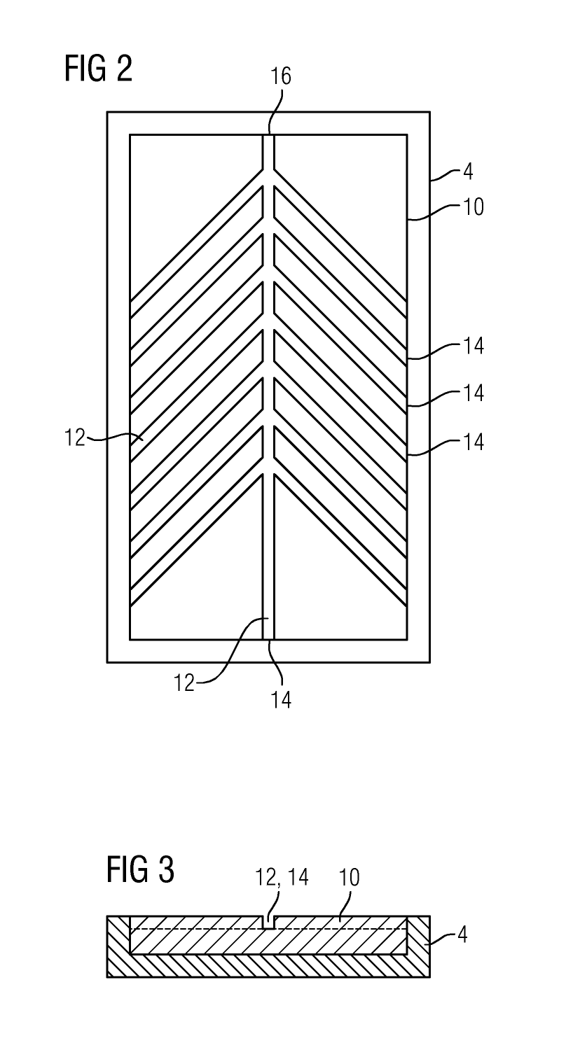

FIG. 2 depicts, schematically and by way of example, a patient couch support 10 with a herringbone pattern depicted from above. The patient couch support 10 (e.g., with a rectangular footprint), includes a surface for receiving an examination object (not shown) and is disposed on a patient couch 4. The patient couch support 10 further includes a number of channels 12 for carrying a fluid (e.g., a gaseous fluid, such as air) that run partly within the patient couch support 10. The channels 12 run in straight lines in the shape of a herringbone pattern. The channels 12 run in straight lines with a wider central channel, similar to a backbone, and a number of narrow ray-shaped side channels similar to fish bones. The channels 12 include openings that are disposed in an edge area of the surface of the patient couch support 10. The openings depicted in FIG. 2 include the lower part and the side parts for inlet openings 14 for supplying the fluid, and the openings on the top side for outlet opening 16 for taking away the fluid. An air blower may blow air for cooling an examination object supported on the patient couch support 10 in the direction of the inlet openings 14 (e.g., within an examination or of an MRT device) and to flow through the channels 12 out of the outlet opening 16. As an alternative, the sheet may be taken away “passively” with air circulation constantly present flowing through the channels 12. In such cases, the position of the inlet openings 14 and the outlet opening 16 may vary. In an example, the patient couch support 10 is provided as a foam material, into which the channels 12 are formed. The raised structures may help to support the examination object while the cutouts carry the heat-removing fluid. The patient couch support 10, provided as a foam material and with a channel structure, may use a stiffer foam material than a patient couch support without a channel to avoid an impression of the material that may constrict the channel structure when subjected to loading by the examination object.

FIG. 3 depicts, schematically and by way of example, the patient couch support 10 from FIG. 2 in a cross-sectional diagram. The patient couch support 10 is able to be fitted into the patient couch 4 to make a form fit. FIG. 3 depicts the central channel 12 with an inlet opening 14. The channel 12 is open to one side to accept an examination object (not shown). The channel 12 may be provided as an open gulley, a groove or trough.

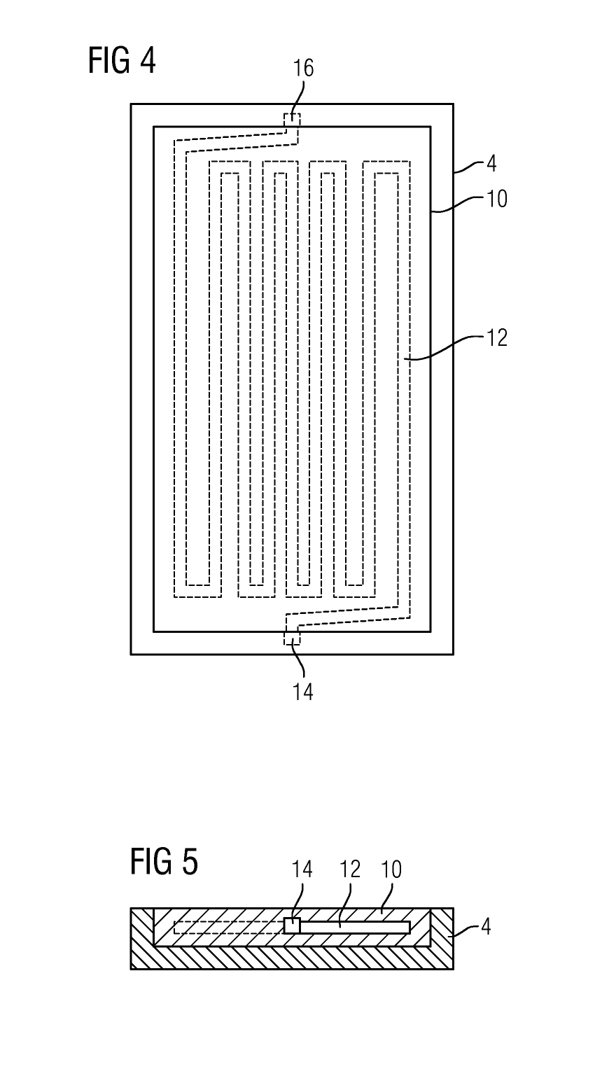

In FIG. 4 depicts, schematically and by way of example, a patient couch support 10 with a channel 12 running in a serpentine shape is disposed on a patient couch 4 in a view from above. In this exemplary embodiment that represents a patient couch support 10 for accepting a spinal column of a human patient, the channel 12 has just one inlet opening 14 for delivering a fluid and just one outlet opening 16 for taking away the fluid. Thus, the patient couch support 10 includes one channel 12. The channel 12 has a serpentine shape (e.g., the channel 12 runs in loops over the surface of the patient couch support 10). The serpentine shape enables a large surface of the patient couch support 10 to be covered with the one channel that may increase the efficiency of the heat removal of the cooling. In this exemplary embodiment, the inlet opening 14 is provided to receive the fluid from a pumping device and the outlet opening 16 is embodied to output the fluid to a pumping device. For example, the openings may have a sleeve, a screw, push-in or bayonet connections. If the fluid involves a liquid, the pumping device may involve a known pump. For a gaseous fluid, such as air, the pumping may involve a ventilator or a fan. The density of the channel runs of the channel 12 over the surface of the patient couch support 10 is predetermined such that, in the area of the spinal-column, a higher number of channel runs (e.g., channel sections of the channel 12) per length cross-section is provided than in the edge areas.

FIG. 5 depicts, schematically and by way of example, patient couch support 10 from FIG. 4 in a cross-sectional view. The patient couch support 10 is fitted into a patient couch 4. In this embodiment, the channel 12 is disposed inside the patient couch support 10. The arrangement inside the patient couch support 10 may guarantee that the channel 12 is only open at the inlet opening 14 and at the outlet opening not visible in FIG. 5, or is designed to deliver and/or remove the fluid. Because heat may only be transferred thereby through the material of the patient couch support, even if it is a thin design, a flow of the fluid may be forced through the channel 12 (e.g., by a pumping).

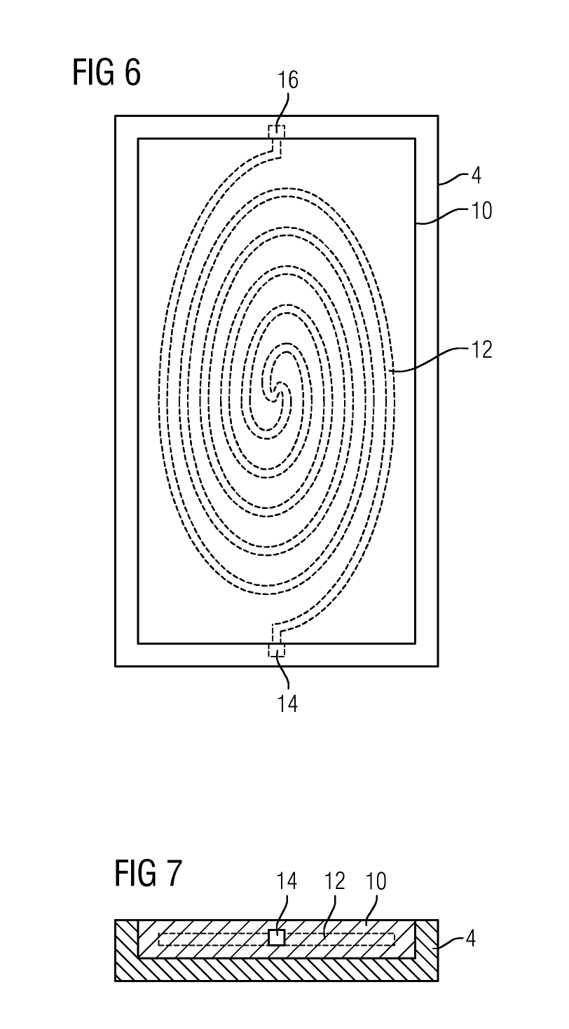

In FIG. 6, schematically and by way of example, a patient couch support 10 with a channel 12 running in a spiral shape, disposed on a patient couch 4, is depicted in a view from above. The spiral shape enables a large surface of the patient couch support 10 to be covered with the single channel 12. The spiral is fitted into the rectangular footprint of the patient couch support 10, so that the length-to-width ratio is not equal to 1, and the spiral has an elongated effect. because the inlet opening 14 for delivering fluid and the outlet opening 16 for the removal of fluid are disposed in an edge area of the rectangular footprint of the patient couch support 10, the channel 12 turns until the channel reaches a central point inside of the rectangular footprint in a spiral shape and then reverses the direction of rotation to turn out in a spiral shape to an edge area of the rectangular footprint of the patient couch support 10.

FIG. 7 depicts, schematically and by way of example, the patient couch support 10 from FIG. 6 in a cross-sectional view. The patient couch support 10 is fitted into a patient couch 4, the channel 12 is disposed inside the patient couch support 10, the inlet opening 14 serves to deliver a fluid and an outlet opening (not shown) serves to take away the fluid.

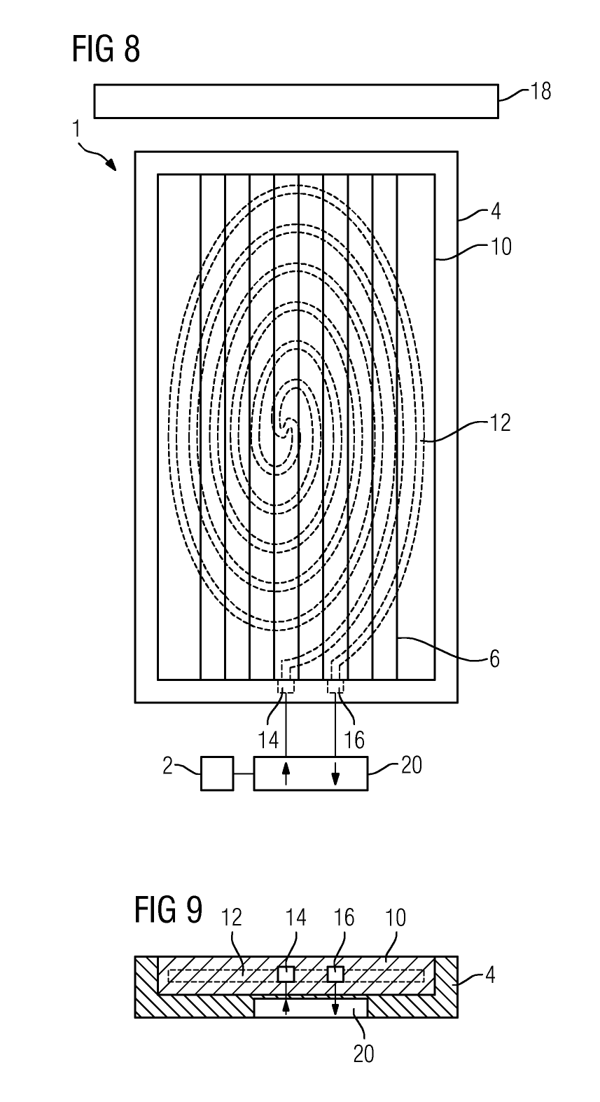

In FIG. 8, schematically and by way of example, a system 1 for dissipating heat of an examination object with a patient couch support 10, with a channel 12 running in a spiral shape that is fitted into a patient couch 4, is depicted in a view from above. The patient couch support 10 includes a local magnetic resonance coil device 6 designed as a receive coil (e.g., as a spinal column coil). The magnetic resonance coil device 6 depicted is to be understood as a symbolic representation because receive coils typically include a number of partly overlapping, round, oval or rectangular elements.

The channel 12 runs in a spiral shape similar to that depicted in FIG. 7, however the inlet opening 14 and the outlet opening 16 are disposed in the edge area of the same side of the surface of the patient couch support 10. Less space is needed for connections to the inlet opening and to the outlet opening. The inlet opening 14 and the outlet opening 16 are on a side which is the furthest away from the examination bore opening when the couch is moved into the examination bore 18. This arrangement may reduce the danger of a collision with edge areas of the examination bore 18. The system 1 further includes a pumping device 20 for delivering a fluid, here a liquid, into the inlet opening 14. The liquid fluid is taken away again by the pumping device 20 at the outlet opening 16 (e.g., a cooling circuit may be formed). To set the cooling effect, a throughput (e.g., in liters per hour) and/or the temperature (e.g., in degrees Celsius) may be specified. Temperature and/or throughput may be set manually (e.g., by a user), or automatically, as indicated in this exemplary embodiment, by an adjustment device 2, such as a computer. When the adjustment device 2 is used, a regulation using a measurement of an actual temperature, a specification of the required temperature and a regulation strategy for reaching the required temperature may be used.

FIG. 9, schematically and by way of example, presents the patient couch support 10 from FIG. 8 in a cross-sectional view. The patient couch support 10 is fitted into a patient couch 4, the channel 12 is disposed inside the patient couch support 10, the inlet opening 14 serves to deliver a fluid and the outlet opening and the outlet opening 16. The outlet opening 16 is disposed on the same side as the inlet opening 14, and serves to take away the fluid. A pumping device 20 is disposed in the patient couch 4.

In an embodiment, channels are let into the upper area of a patient couch support that make air circulate without using a fan. The air circulation enables a cooling effect to be achieved. If the channels are provided in a foam material, a material with suitable stiffness may be used to prevent or reduce a compression of the channels when subjected to a load by an examination object. The shape of the course of the channels may be selected as a function of the position of the examination object and of the examination area to be imaged.

By using a fan or a pump, air or a coolant may be carried through one or more tubes that run within a patient couch support. For example, throughput of the coolant may be adjusted continuously or in stages (e.g., by an operator or automatically), with temperature sensors in the patient couch support that may measure the actual current temperature of the patient couch support. The shape or the course of the channels may be selected as a function of the position of the examination object, of the examination area to be imaged or of the heat sources to be expected (e.g., in so-called hotspots).

One or more of the embodiments of the patient couch support may achieve a temperature felt to be more comfortable by the examination object at the contact surface to the patient couch support and may reduce moisture at this contact surface.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A patient couch support for dissipating heat, the patient couch support comprising:
a surface configured to accept an examination object;
at least one channel configured to carry a fluid running at least partly inside the patient couch support; and
wherein the at least one channel comprises at least one inlet opening for delivering the fluid and at least one outlet opening for taking away the fluid.

2. The patient couch support of claim 1, wherein the at least one inlet opening and the at least one outlet opening are each disposed in an edge area of the surface of the patient couch support.

3. The patient couch support of claim 2, wherein the at least one channel is formed by cutouts in a foam.

4. The patient couch support of claim 3, wherein a density of channel runs of the at least one channel running over the surface of the patient couch support is predetermined.

5. The patient couch support of claim 4, wherein the at least one channel has a serpentine shape.

6. The patient couch support of claim 4, wherein the at least one channel has a flat spiral shape.

7. The patient couch support of claim 4, wherein the patient couch support has a plurality of channels, wherein the channels at least partly run in straight lines.

8. The patient couch support of claim 7, wherein the channels have a herringbone pattern.

9. The patient couch support of claim 4, wherein the at least one inlet opening is configured to accept the fluid from a pumping device, wherein the at least one outlet opening is configured to output the fluid to the pumping device, or wherein the at least one inlet opening is configured to accept the fluid from the pumping device and the at least one outlet opening is configured to output the fluid to the pumping device.

10. The patient couch support of claim 4, wherein the at least one channel configured to accept the examination object is open to one side.

11. The patient couch support of claim 4, wherein the at least one channel is disposed inside the patient couch support.

12. The patient couch support of claim 11, wherein the at least one channel has only one inlet opening for delivering the fluid and only one outlet opening for taking away the fluid.

13. The patient couch support of claim 12, wherein the inlet opening and the outlet opening are disposed in the edge area of the same side of the surface of the patient couch support.

14. The patient couch support of claim 1, wherein the patient couch support includes a local magnetic resonance coil device.

15. The patient couch support of claim 14, wherein the local magnetic resonance coil device is a spinal column coil or a head coil.

16. A patient couch for dissipating heat of the patient couch support, the patient couch comprising:
a patient couch support comprising:
a surface configured to accept an examination object;
at least one channel configured to carry a fluid running at least partly inside the patient couch support; and
wherein the at least one channel comprises at least one inlet opening for delivering the fluid and at least one outlet opening for taking away the fluid.

17. A system for dissipating heat of the patient couch support, the system comprising:
a patient couch comprising:
a patient couch support comprising:
a surface configured to accept an examination object;
at least one channel configured to carry a fluid running at least partly inside the patient couch support;
wherein the at least one channel comprises at least one inlet opening for delivering the fluid and at least one outlet opening for taking away the fluid;
a pumping device for at least delivering the fluid into the inlet opening; and
wherein the fluid has a predeterminable throughput, a predeterminable temperature, or a predeterminable throughput and predeterminable temperature.
